(19) United States
(12) Patent Application Publication
(10) Pub. No.: US 2010/0076381 A1
(43) Pub. Date: Mar. 25, 2010

(54) TROCAR VALVE SEALS

(76) Inventor: Jesper Schantz Simonsen, Copenhagan (DK)

Correspondence Address: WINSTON & STRAWN LLP;PATENT DEPARTMENT, 1700 K STREET, N.W., WASHINGTON, DC, 20006, US

(21) Appl. No.: 12/517,332

(22) PCT Filed: Dec. 5, 2007
(86) PCT No.: PCT/IB07/54934

§ 371 Date: Jun. 2, 2009

(30) Foreign Application Priority Data
Dec. 5, 2006 (DK) PA 2006 01598

(51) Int. Cl. A61B 17/34; A61M 39/06

(52) U.S. Cl. 604/167.02; 604/256

(57) ABSTRACT

A valve seal for a trocar cannula having a distal end and an opposing proximal end with an enlarged section for accommodating at least a part of a valve seal. The valve seal has a tubular neck part defined by a first circumferential wall, an instrument leaving end and an opposing instrument receiving end, the instrument receiving end is provided with a first diaphragm valve and a circumferential flange connecting the tubular neck part with a substantially concentric second circumferential wall surrounding at least a part of the first circumferential wall to define a gap between said substantially concentric circumferential walls, at a distance from the first diaphragm valve the first circumferential wall of the tubular neck part is axially split up into an exterior tubular neck part and a substantially concentric interior tubular neck part provided with a second diaphragm valve, the interior tubular neck part terminating a distance from the instrument leaving end. The valve seal is moulded in its final form. The valve seal has at least three valves, which enhance sealing capability, and the design of the valve seal is suitable for trocars with cannulas of various diameters.

The present invention relates to a blank for the manufacturing of a valve seal for a trocar cannula having a distal end and an opposing proximal end with an enlarged section for accommodating at least a part of the valve seal.

The present invention also relates to a method of making a valve seal from the blank, and to valve seals for trocars.

A trocar is a sharp-pointed surgical instrument fitted with a cannula, a valve and a stylet. By means of the stylet the cannula is inserted into a body cavity to provide e.g. a drainage outlet, or to provide an access port for easy exchange of endoscopic instruments during endoscopic surgery. Often a trocar is used during a laparoscopic procedure to create an entry site for various kinds of viewing apparatus or surgical instruments. Once the trocar is inserted into the body the valve closes and seals around the instrument to assist in preventing both unintentional and uncontrolled escape and leakage of the liquids or gases, which are naturally occurring in the body cavity or are supplied in the surgical procedure as insufflation gases or irrigation liquid.

Effective sealing around the instrument or tube inserted through the valve seal of the trocar is essential for the surgeon during the surgical procedure. For example, an insufflated cavity needs to be kept distended during the entire procedure. If the valve seal leaks too much the distension may be insufficient or discontinue with the result that the inflated cavity unintentionally collapses. A further problem when using most known trocars is the fact that the appropriate diameters of the various apparatus' or instruments the surgeon need to use must be within a very narrow margin in order not to compromise the sealing. Hence, once the trocar is inserted the surgeon's freedom to operate through a trocar is limited by the dimensions of the trocar and the sealing capability of the specific valve structure of the selected trocar.

In order to remedy these disadvantages and problems various attempts have been made to develop new and more effective valve seals and valve seals assemblies for trocars.

From U.S. Pat. No. 5,727,770 is known an elastomeric double valve sealing device. The valve seal has two valves; a conical split valve to seal the cannula when no instrument is present, and a diaphragm-type valve for sealing when an instrument is inserted. If an instrument having a large diameter is inserted through such a valve seal the conical split valve is deformed to an extent which does not allow it to regain a closed position and liquid and gases can escape through the not fully closed valve when one or more instruments are manoeuvred both axially and radially in the valve seal.

International patent application no. WO 2005/013807 discloses another valve for a trocar. This known valve closes around an inserted instrument by means of a “duck bill” valve. However, the two converging flaps of such a “duck bill” valve is only able to seal to a limited degree around the inserted instrument. Especially when used with instruments having a diameter greater than 5 mm, sealing is inadequate.

In order to optimise sealing capability around instruments of various diameters the trocar valve known from U.S. Pat. No. 5,385,553 has been developed. This known trocar valve has a floating septum with a plurality of pivoting levers and mechanically cooperating components, which makes the valve a rather complicated construction susceptible to malfunction with the risk that the surgeon is unable to retract the inserted instrument from the trocar cannula.

It is a first aspect according to the present invention to provide a simple, inexpensive embodiment of a blank, which can be transformed into a valve seal for a trocar.

It is a second aspect according to the present invention to provide a valve seal for a trocar, which fits firmly on the trocar cannula, seals effectively around an inserted instrument, and closes effectively when no instrument is inserted.

It is a third aspect according to the present invention to provide a valve seal for a trocar, which can be used with surgical instruments having different diameters.

It is a fourth aspect according to the present invention to provide a valve seal for a trocar, wherein an inserted surgical instrument can be manoeuvred and displaced both radially and axially in the valve seal without resultant leakage of gases or liquid from the accessed body cavity.

It is a fifth aspect according to the present invention to provide a valve seal for a trocar, which valve seal has a lower surface friction drag than known valve seals.

It is a sixth aspect according to the present invention to provide a trocar, which can be used with surgical instruments provided with surgical tools with bend or hook-like operational parts which may catch the valve seal during withdrawal of the instrument.

The novel and unique feature whereby this is obtained is the fact that the blank has a tubular neck part defined by a first circumferential wall, an instrument leaving end and an opposing instrument receiving end, the instrument receiving end is provided with a first diaphragm valve and a circumferential flange via which the tubular neck part extends in an axial direction into a tubular head part defined by a second circumferential wall, said tubular head part has an interior diameter, which is greater than the exterior diameter of the tubular neck part.

As used herein the term “trocar” is generally used to refer to the entire assembly of a trocar cannula with a valve seal or valve seal assembly and a trocar stylet. The trocar cannula is introduced in the body by means of the trocar stylet inserted in the bore of the trocar cannula. The trocar stylet is designed with a tip, e.g. in the shape of a three edged pyramid or a flat two edged blade or a blunt end, which tip serves for facilitating penetration of the body wall. Once the stylet is retracted a surgical instrument can be introduced and used inside the body. Surgical instruments include but are not limited to e.g. surgical cutting and cauterization electrodes having surgical tips of various shapes, such as L, J, needle or spatula, scissors, forceps, clip appliers, graspers and suction and irrigation equipment.

As used herein the term “blank” is to be understood as an object or preform ready to be made into an intermediate object or a ready-for-use form. In the context of the present application “blank” is used for a preformed body or element, which can be transformed into a valve seal for a trocar.

Within the scope of the present application the term “diaphragm valve” is used to define an annular partition wall, a ring, a plate or any similar annular body having a central aperture, and which is used to limit an aperture of another object or passageway, e.g. to reduce the accessibility of gas or air passage around the instrument.

A blank having the above configuration and interdependent dimensions can be transformed into a valve seal for a trocar just by simply turning the tubular head part inside out so that the exterior face of the tubular head part is brought to face the exterior face of the tubular neck part. The turning of the tubular head part exposes the first diaphragm valve and the circumferential flange surrounding the first diaphragm valve, so as to create a valve seal with an easy accessible first valve opening for an insertable object such as e.g. a surgical instrument, apparatus or tube.

The turning of the tubular head part inside out creates a collar or sleeve for mounting of the valve seal in and around the enlarged section of the trocar cannula. The turning is especially easy to perform if the second tubular head part extends axially from the first circumferential wall at a pivot joint.

In a simple and inexpensive embodiment for a blank according to the present invention, the pivot joint may be created by simply decreasing the wall thickness of the circumferential flange towards the pivot joint. The decreased wall thickness at the pivot joint provides the blank with the capability of almost by itself immediately after turning of the tubular head part to assume the final structural valve seal form, which is required for the turned or inverted tubular head part of the valve seal to be mounted around the enlarged section of the trocar cannula when the tubular neck part of the valve seal are inserted into the bore of the trocar cannula. In addition, by decreasing the wall thickness at the pivot joint no material from the second circumferential wall is left in the way to obstruct the fitting of the valve seal on the cannula, and the inner side of the turning can be made with a smooth abutment surface, so that the valve seal may be mounted firmly on the outmost edge on the enlarged section of the trocar cannula. The pivot joint may be made so that the collar may be parallel to the tubular neck part, however within the scope of the present invention the thickness of the pivot joint may also be selected to allow the free end of the tubular head part to converge against the tubular neck part, to provide a valve seal which fits even tighter around the trocar cannula.

In an expedient embodiment of a blank according to the present invention for making a valve seal the first circumferential wall of the tubular neck part can, at a distance from the first diaphragm valve, be axially split up into an exterior tubular neck part and a substantially concentric interior tubular neck part provided with a second diaphragm valve terminating a distance from the instrument leaving end. This distance may e.g. be half or less than the longest length of the tubular neck part and smaller than the shortest length of the tubular neck part.

A trocar, which is equipped with the tubular neck part of a trocar valve located inside the enlarged section of the trocar cannula, the collar of the trocar valve is made by e.g. turning the tubular head part inside out surrounding at least the enlarged section of the trocar cannula, is in surgical use introduced through the abdominal wall into the body of a patient at a desired location. Subsequently, the surgical instrument is inserted in the valve seal through a central aperture in the first diaphragm valve. The surgeon now begins to move the tip of the instrument around to reach a certain target inside the body. The object of the surgeon may e.g. be to inspect, to aspirate liquid, to perform a surgical procedure, or to take a biopsy. When the instrument is manipulated in the valve seal the instrument frequently collides with the first circumferential wall, which is compressed against at least segments of the rigid wall of the trocar cannula. The first circumferential wall cannot yield beyond the limits defined by the rigid wall of the trocar cannula, and the diameter of the usable instrument is limited by the dimensions of the trocar once the trocar is inserted. If too large instruments are used they may get stuck in the valve seal. Another risk is, that because the first circumferential wall has no yielding space in response to compression, the valve seal including the first diaphragm valve may be deformed and drawn down into the cannula resulting in that the first diaphragm opens occasionally. When a second diaphragm valve is provided at an interior tubular neck part, the valve seal is able to respond to the movements of the instrument, and to sustain effective sealing around the instrument during the whole surgical procedure. Hence, a valve seal having two diaphragm valves according to the invention provides the surgeon with a high degree of freedom to move an instrument inserted in the valve seal without constantly worrying about leakage. Even though the inserted part of the instrument is moved considerably around inside the body the annular gap between the interior and exterior tubular neck parts allows accommodation of the interior tubular neck part which is able to yield into the gap if displaced. As a result of the gap, the valve seal according to the present invention is less sensitive to contact with the instrument. The inserted instrument can be moved considerably both laterally and longitudinally without compromising the sealing around the instrument and the valve seal can be used with instruments up to at least 12 mm diameter.

When at least one of the first and second diaphragm valves have an annular section of increased thickness and/or a bead along or surrounding the perimeter of its respective valve opening the central opening of any diaphragm valve may have a central aperture with increased thickness. As a result the remaining part of the diaphragm valve is thin and more inclined to yield in response to movement of the instrument during surgery than a diaphragm valve having the same overall thickness. In the embodiment where a valve opening has been given both an annular section of increased thickness and a bead concentric arranged around and along, respectively, the perimeter of the valve opening, a high degree of radial flexibility and deflection of the opening can be maintained in response to instruments of various cross-sections or diameters, as well as the radial pressure force exerted on the instrument along the perimeter of the opening to obtain sealing is kept optimum.

Even further degree of yielding capability, in particular in response to radially movement, is obtained if the second diaphragm valve tapers towards the instrument receiving end. The free annular space inside the interior tubular neck around the upwards tapering central aperture of the second diaphragm valve provides a radial clearance similar to a bellow when the instrument is moved. Accordingly, the second diaphragm valve is able to follow the instrument when moved and sealing is not affected.

The exterior face of the second circumferential wall may expediently be provided with at least one coupling rib or thread for engagement with the enlarged section of the cannula.

If the instrument leaving end of the exterior tubular neck part is sloped the valve seal may advantageously be provided with a third valve in an easy manner. The opening may be covered by an axially displaceable resilient flap on the valve seal. The flap may be a separate object or an integrated part of the valve seal. When the instrument is inserted the valve may be displaced to open the third valve, and when the instrument is removed the resiliency of the flap causes the flap to cover and close the instrument leaving end.

As described above a valve seal can be made from the blank according to the present invention in the above mentioned method in which the second circumferential wall of the tubular head part is pivoted about the pivot joint to surround at least a part of the second circumferential wall of the tubular neck part. The method is particularly easy to carry out if the blank is moulded of an elastic material having a memory shape.

Within the scope of the present invention alternatively, the valve seal can also be moulded in its final form. This inventive valve seal having a final form corresponds in a first embodiment according to the present invention substantially to the valve seal made from the blank and has the same advantages.

Accordingly, the prefabricated valve seal has a tubular neck part defined by a first circumferential wall, an instrument leaving end and an opposing instrument receiving end, the instrument receiving end is provided with a first diaphragm valve and a circumferential flange connecting the tubular neck part with a substantially concentric second circumferential wall surrounding at least a part of the first circumferential wall to define a gap between said substantially concentric circumferential walls, at a distance from the first diaphragm valve the first circumferential wall of the tubular neck part is axially split up into an exterior tubular neck part and a substantially concentric interior tubular neck part provided with a second diaphragm valve, said interior tubular neck part terminates a distance from the instrument leaving end.

Just as the blank, the prefabricated valve seal may be designed so that at least one of the first and second diaphragm valves has an annular section of increased thickness and/or a bead along or surrounding the perimeter of its respective valve openings. The face of the second circumferential wall may face towards the tubular neck part, the second circumferential wall may be provided with at least one coupling rib or thread, and the instrument leaving end of the exterior tubular neck part may be sloped. The advantages of these features are described above for the blank.

Preferably, the valve seals according to the present invention is provided with a third valve, constituted by the sloped instrument leaving end in cooperation with a hinged or integrated flap which is displaceable to open the instrument leaving end of the valve seal by means of an instrument introduced in the valve seal and to substantially close the instrument leaving end when the instrument is retracted from the valve seal. A third valve further ensures the sealing capability of the valve seal.

When the valve seal is made from the blank the hinged flap is especially easy to mount on the blank prior to turning of the tubular head part. If the valve seal is prefabricated into its final shape a hinged flap may simply be suspended around or hinged to the tubular neck part. Alternatively, the flap may be an integrated part of the blank or the moulded valve seal.

For use with surgical instruments having hooks or barbs which may catch the flap upon retraction of the instrument the flap may be provided with a convexity facing the instrument leaving end of the valve seal, said convexity forces the hooks or barbs away from the flap so that the hook or barb not catches the flap.

In a preferred embodiment according to the present invention the valve seal may, irrespectively of the valve seal is made from the blank or is made in its final form, further comprise means, permanently or detachable, for reducing friction between the valve seal and the instrument when the instrument is moved in and out of the valve seal. The friction reducing means may be fully inserted between the first diaphragm valve and the second diaphragm valve, or the first diaphragm valve may be constituted partly by means of the friction reducing means, in which case only a part of the friction reducing means is inserted into the valve seal.

Advantageously, the means for reducing friction is a cup, preferably a cup having a cylindrical bottom part extending at least partly into the bore of the tubular neck part, wherein at least the bottom part is split-up. The split-up bottom part of the cup preferably may rest on or abut the second diaphragm valve and assists in preventing the second diaphragm valve from being drawn upwards upon retraction of the instrument.

In a preferred embodiment for a friction-reducing cup especially suited for use in the valve seal according to the present invention the bottom part of the cup may extend axially via a central part into a circumferential, radially extending flange, which overlays the first circumferential flange of the instrument receiving end of the valve seal. This flange may advantageously serve to retain the friction-reducing cup in place and secure that the cup is not displaced in the axial direction towards the instrument leaving end in response to axial and radial movement of an instrument inserted into the valve seal.

If the smallest internal diameter of at least a first axial section of the central part closest to the bottom part is smaller than the largest internal diameter of the bottom part itself the part of the cup protruding inside the bore of the tubular neck part is configured substantially as an hourglass with a flange. When an instrument is inserted the upper part of the hourglass serves as a guiding funnel for an instrument and the constriction of the hourglass advantageously serves for controlled further guidance during advancing the tip of the instrument towards the instrument leaving end.

The cup may be given an exterior design in which the smallest external diameter of at least the first axial section of the central part closest to the bottom part is smaller than the largest external diameter of the bottom part itself. This geometrical shape and design provides the bottom part of the cup with an exterior radial extent in the area above the second diaphragm valve allowing the split-up bottom part to flex radially inside the tubular neck part to enable smooth manoeuvring of instruments of different designs and diameters during the surgical procedure, and flex axially in response to axial displacement of the instrument to avoid that the tool at the tip fatally grasps the cup and withdraws the cup or is stuck inside the cup with the inherent result that the use of the trocar needs to be interrupted.

I a preferred embodiment the means for reducing friction may be a cup comprising a first part having at least a first bottom part and a first central part, and a second part having at least a second bottom part and a second central part, wherein the first bottom part is insertable, radially, axially or both, into the second bottom part or vice versa. In this embodiment the bottom part is split into two circumferentially overlapping bottom parts, the first bottom part and the second bottom part. The overlapping relationship provides a highly flexible passageway for the instrument. The size of the overlap may advantageously be selected to that the passageway maintains annularly closed during operation, replacement or removal of any instrument.

Preferably at least the second bottom part has a recessed interior annular face for accommodating and annularly surrounding the first bottom part to provide a smooth interior cup face leaving behind no protrusions or gaps which may be grasped by the tool or which may obstruct manoeuvring of the tool.

Preferably, also the first central part may be designed to be insertable into the second central part or vice versa, to provide an even higher degree of splitting and further facilitate smooth passage of instruments having large cross sectional areas or diameters.

In a preferred embodiment the cup may be completely divided into two or more separate parts. In this embodiment the first central part extends into a first flange part and the second central part extends into a second flange part. When this embodiment for a cup is inserted into the tubular neck part tight joining of the first and the second flange parts can be obtained by means of coupling means, such as e.g. key and slot, or any other protrusion fitting into a corresponding cavity for providing annular coupling.

In a second embodiment the two flange parts can be designed as an integral continuous flange, where any or both of the bottom parts and/or central parts are designed as separated parts or flaps suspended on the flange, which separated parts overlap each other when radially displaced towards each other.

When the valve seal or the blank is made of an elastomeric material, e.g. by injection moulding, as an integral unit a valve seal having maximum integrity and flexibility can be produced. In case of a blank, the tubular head part is especially easy to fold around the pivot joint. The elasticity of the elastomeric material facilitates firm, elastic attachment and hold of the valve seals according to the present invention on the enlarged section of the cannula.

A preferred material for moulding the valve seal, the blank and/or the cup is a material having self-lubricating properties, such as a silicone rubber, e.g. a Self-Lube Silicone Rubber which is produced by NuSil Technology USA, 1050 Cindy Lane, Carpinteria, Calif. 93013 USA. By choosing such a material the coefficient of friction between the reciprocating instrument and the valve openings are substantially reduced. A greasing substance, e.g. glycerine, may expediently be used for additional greasing of any part of the valve seal, including but not limited to the cup. Preferably at least the openings defining the passageway for the instrument is additionally greased with glycerine. Alternative glycerine greased greasing rings may be inserted wherever appropriate.

Any of the valve seal, the blank, the friction reducing cup and the flap may be moulded using any suitable conventional techniques, e.g. injection moulding.

The invention further relates to a trocar cannula having a distal end and an opposing proximal end with an enlarged section for accommodating the valve seals according to the present invention and described above.

The trocar cannula may according to the invention be fitted with a fastening ring, having a first end for coupling on the enlarged section of the proximal end to hold the valve seal in place. The opposing second end of the fastening ring may conveniently be used for coupling to an instrument guiding member. Any of the fastening ring and the guiding member may be detachable coupled to each other. Alternatively, the fastening ring and the guiding member may be an integral part, or the fastening ring and the guiding member may even be integral with the enlarged section of the trocar cannula.

The guide member may advantageously encompass a greasing ring, which serves for additional greasing and reduction of frictional drag towards the instrument. The greasing ring may be greased with glycerine.

The enlarged section may be provided with a gas inlet opening, to be opened and closed in order to control supply of an insufflation gas if required.

Minimally invasive surgery typically involves use of multiple trocars and cannulas. The first trocar inserted, or primary trocar, is used to place a trocar cannula through which a laparoscope is inserted to view internal structures. Other, secondary, trocars provide for insertion of other instruments such as biopsy forceps, etc. The primary trocar must typically be inserted using a “blind” puncture or “cut-down”. Before inserting the primary trocar some general surgeons and most gynaecologists prefer, instead of direct entry, to insufflate the abdominal cavity by introducing carbon dioxide gas. The insufflation creates a preliminary pneumoperitoneum, which elevates and holds the abdominal wall away from internal structures to avoid accidents on internal structures not involved.

There is a small risk that certain instrument tips may catch the flap of the third valve, resulting in that the instrument gets stuck and trapped inside the valve. If this situation accidentally occurs, the trocar cannula must be removed or be substituted by a new trocar cannula by repeating the surgical insertion procedure of a new trocar. This is inconvenient to the surgeon, prolong surgical time, the patient suffers and recovery may prolong, and also the surgical costs increase. So in order to avoid that the tools, e.g. an L- or J-shaped electrode tip, grasps the flap when the instrument is retracted, at least an annular part of the enlarged section may be provided with an interior annular wall part for restricting unintentionally return movement of the flap after it has been forced open by the instrument and for restriction radial movement of an inserted instrument. Upon axial forwards movement of the tool on the instrument tip through the valve seal and further into the body the tip forces the flap to open. The flap may by suitable dimensioning, alternatively be pushed into frictional engagement with the interior annular wall part to hold the flap open until closing of the flap is activated, e.g. by actuation from outside the cannula or just by using the pressure of an insufflation gas.

The invention will be explained in greater details below with reference to the accompanying drawing, in which

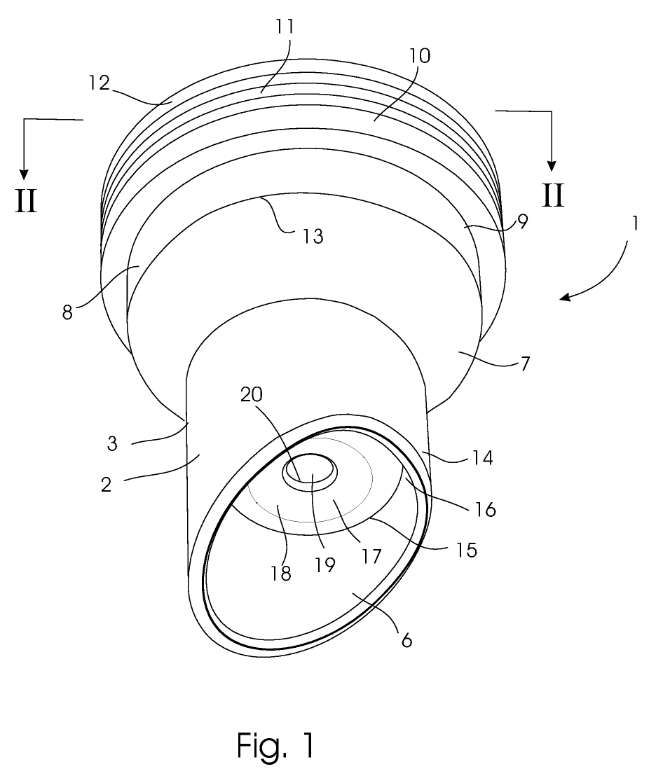

FIG. 1 shows a perspective view, seen from the instrument leaving end, of a blank for a first embodiment of a valve seal according to the present invention,

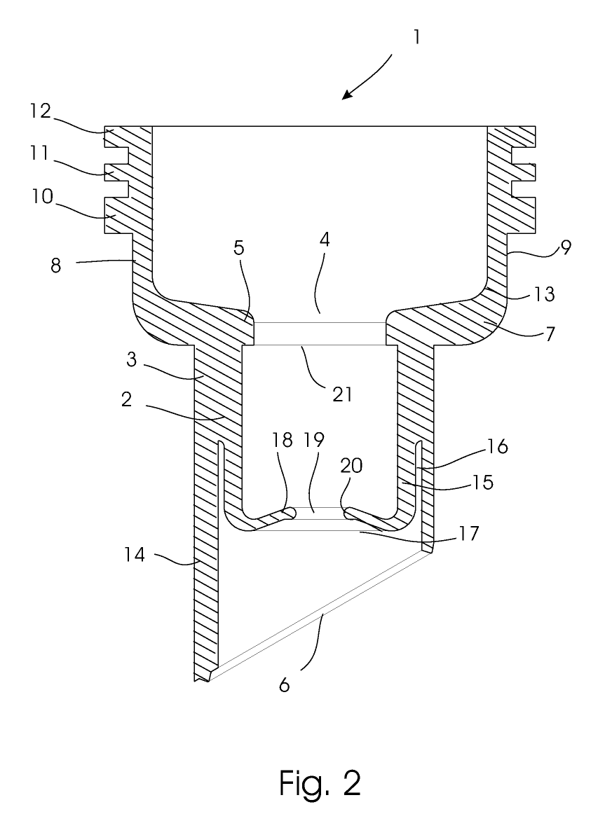

FIG. 2 shows a section taken along the line II-II of the blank seen in FIG. 1,

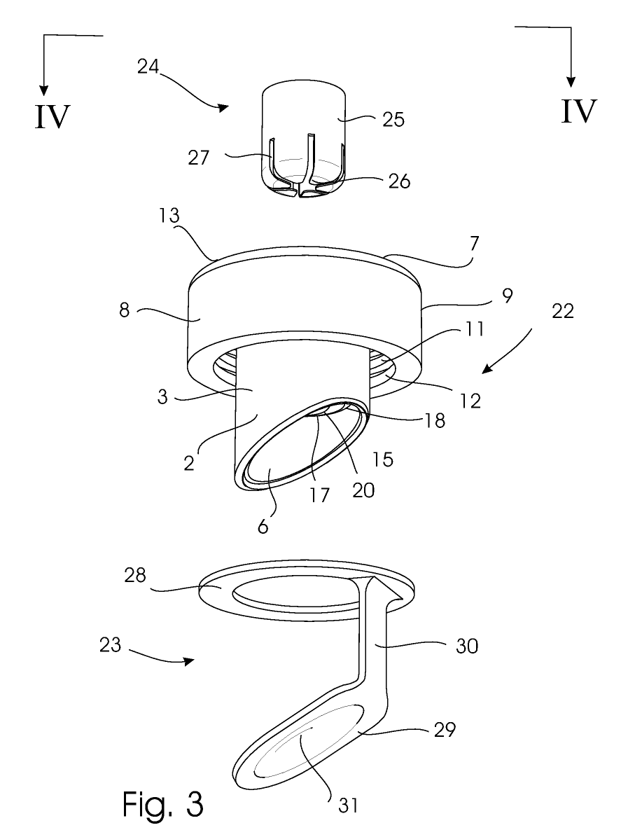

FIG. 3 shows an exploded view of the first embodiment for a valve seal according to the present invention with a first embodiment of a friction reducing means and flap means for closure of the instrument leaving end of the valve seal,

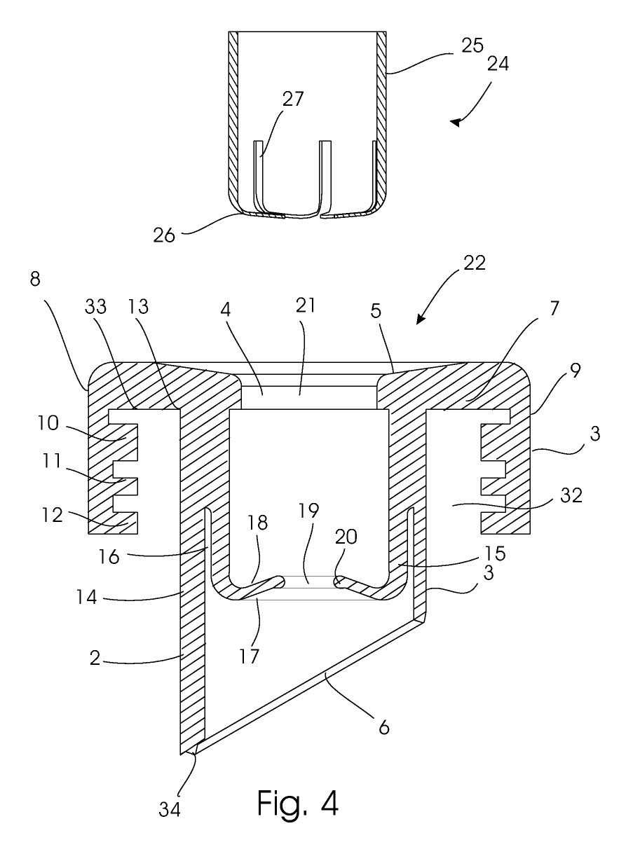

FIG. 4 shows, in enlarged scale, a section of the valve seal and the friction reducing means seen in FIG. 3 taken along the line IV-IV,

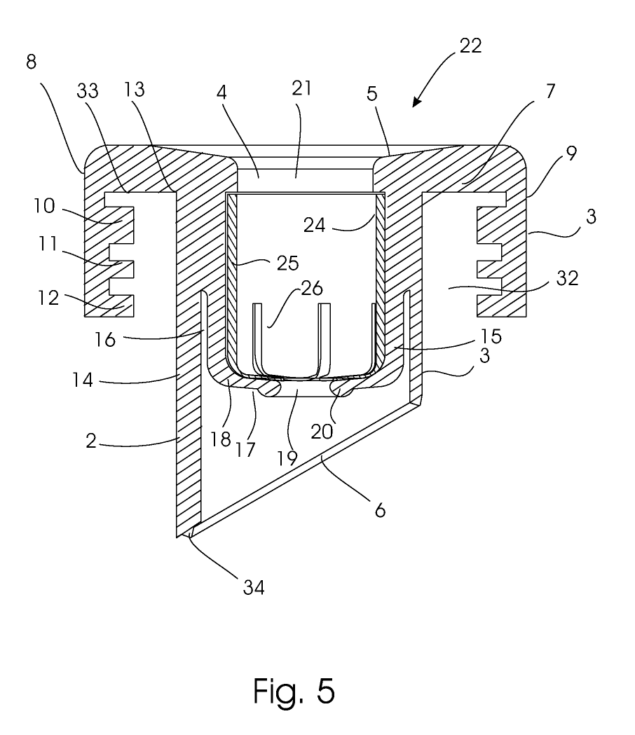

FIG. 5 shows the same with the friction reducing means inserted into the valve seal,

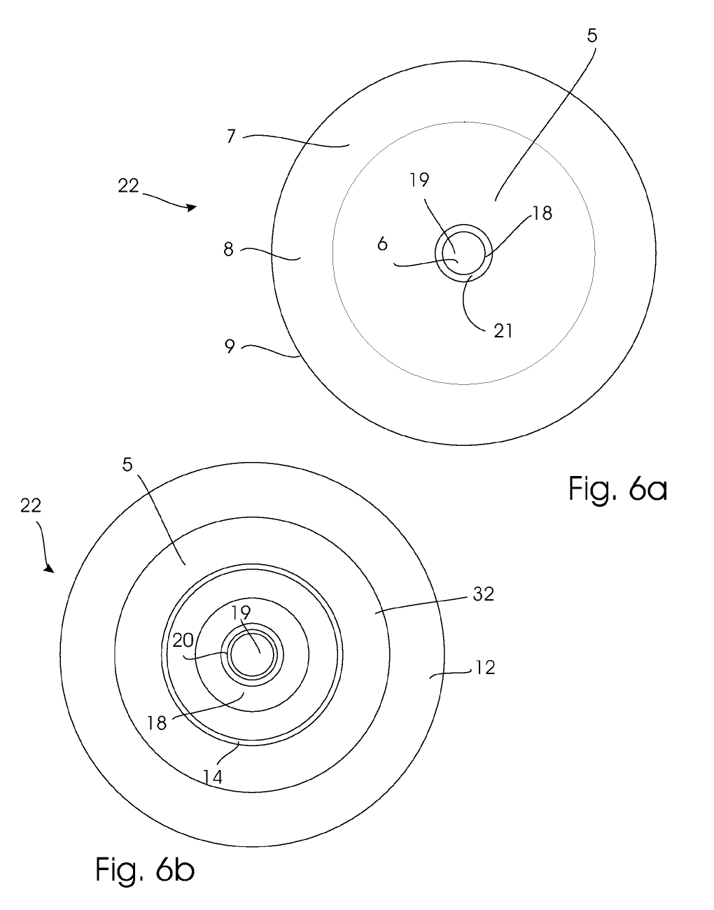

FIG. 6a shows the first embodiment of the valve seal seen from above, that is from the instrument receiving end,

FIG. 6b shows the first embodiment of the valve seal from below, that is from the instrument leaving end,

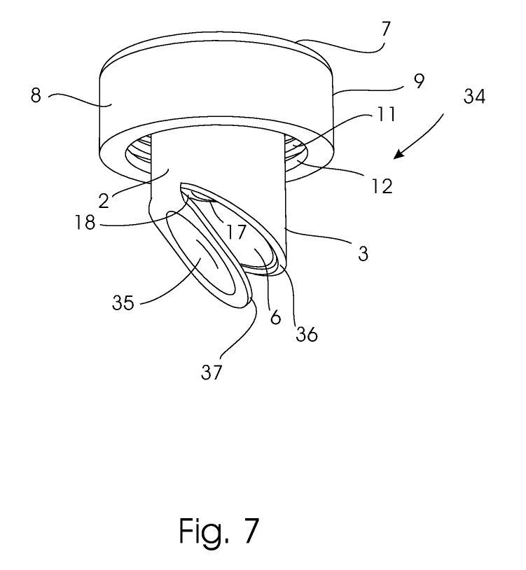

FIG. 7 shows a perspective view of a second embodiment for a valve seal according to the present invention,

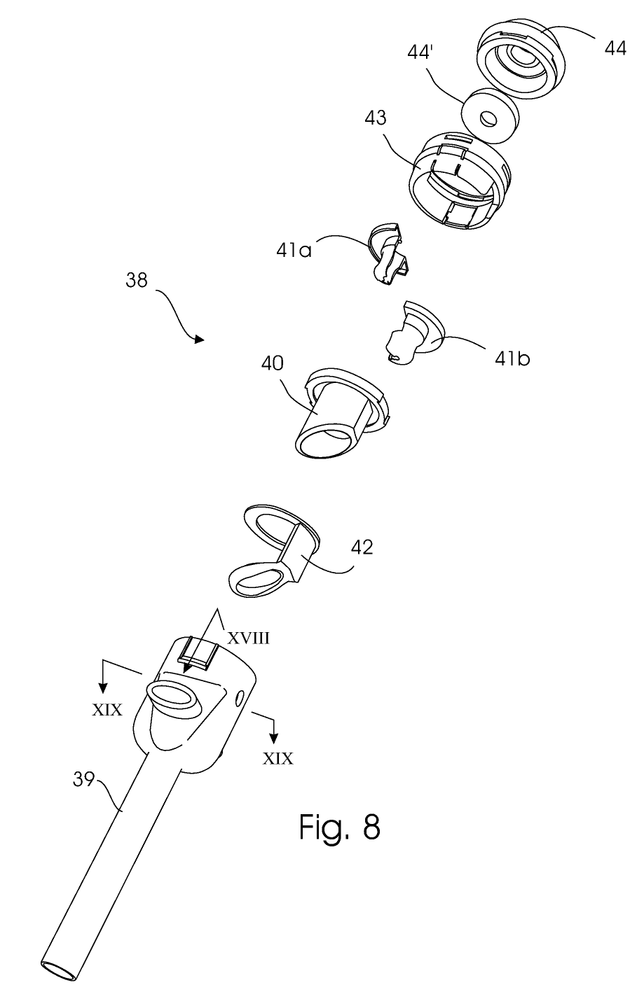

FIG. 8 shows an exploded perspective view of the main parts for a trocar according to the present invention, including a trocar cannula, a third embodiment for a valve seal, a second embodiment for a friction reducing cup, and a modified third valve flap, but without stylet,

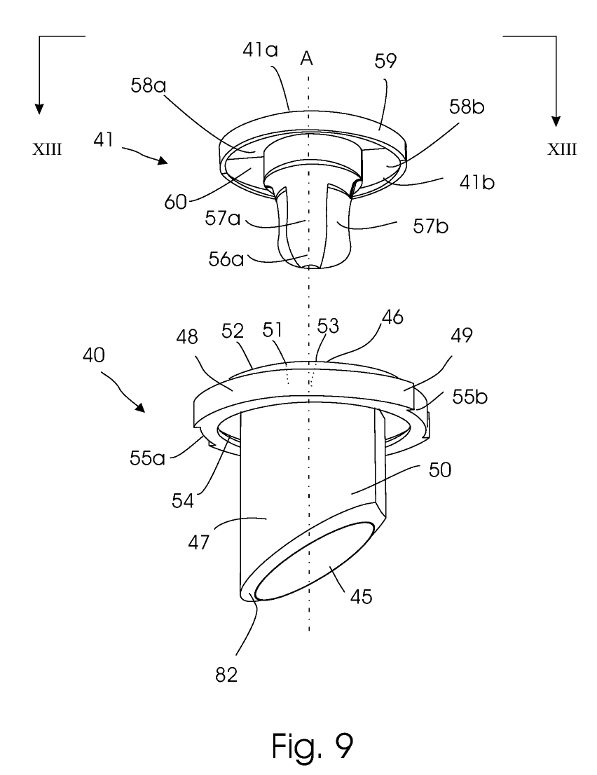

FIG. 9 shows, in an enlarged scale, an exploded view of the third embodiment for a valve seal shown in FIG. 8 with the second embodiment of a friction reducing means and the modified third valve for closure of the instrument leaving end of the valve seal,

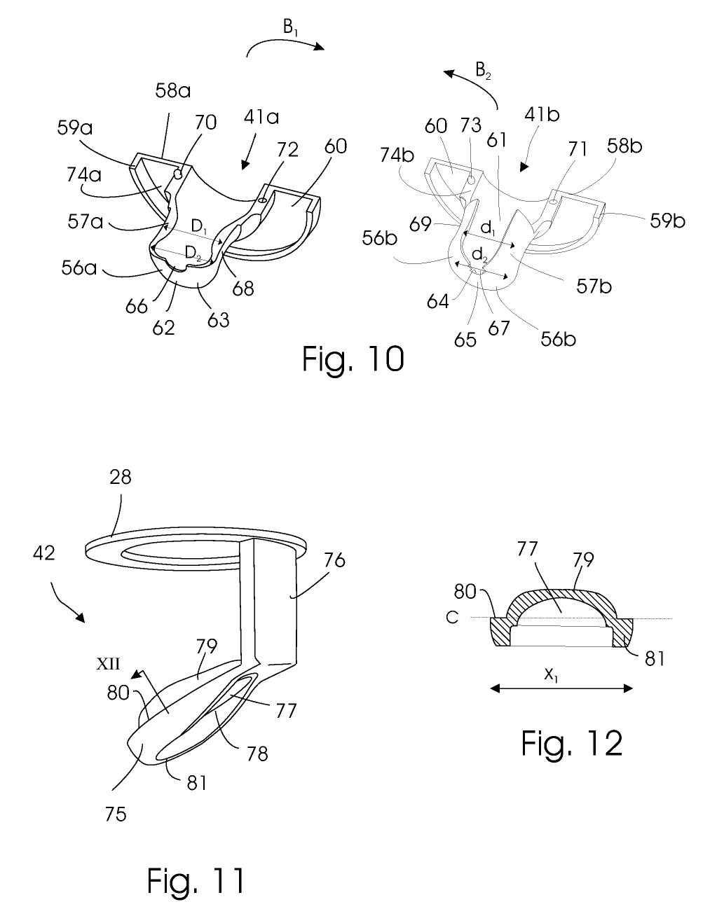

FIG. 10 shows a perspective view, seen from inside oblique from the bottom parts, of the two separate parts defining the second embodiment of the friction reducing cup,

FIG. 11 shows a perspective view of a second embodiment of a flap means for a third valve,

FIG. 12 shows a sectional view taken along line XII in FIG. 11,

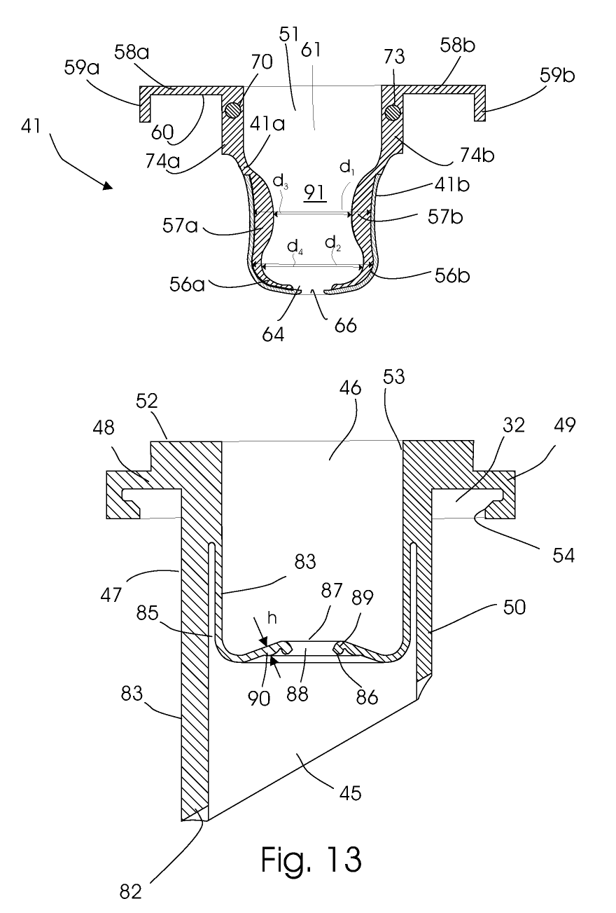

FIG. 13 shows a sectional view taken along line XIII-XIII in FIG. 9 in an enlarged scale,

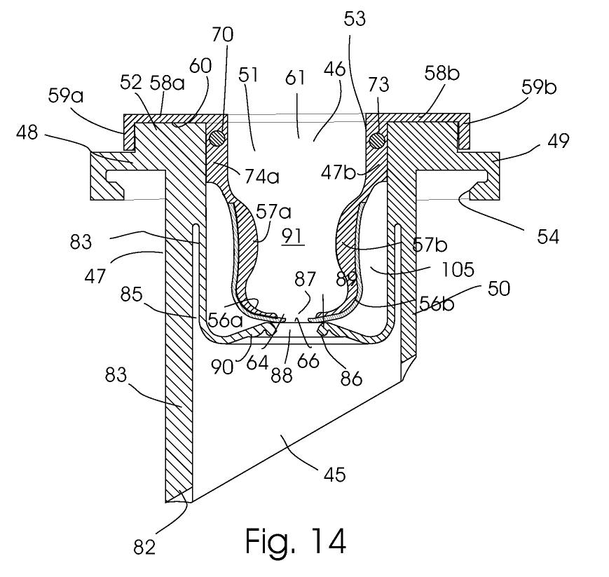

FIG. 14 shows the same in assembled state,

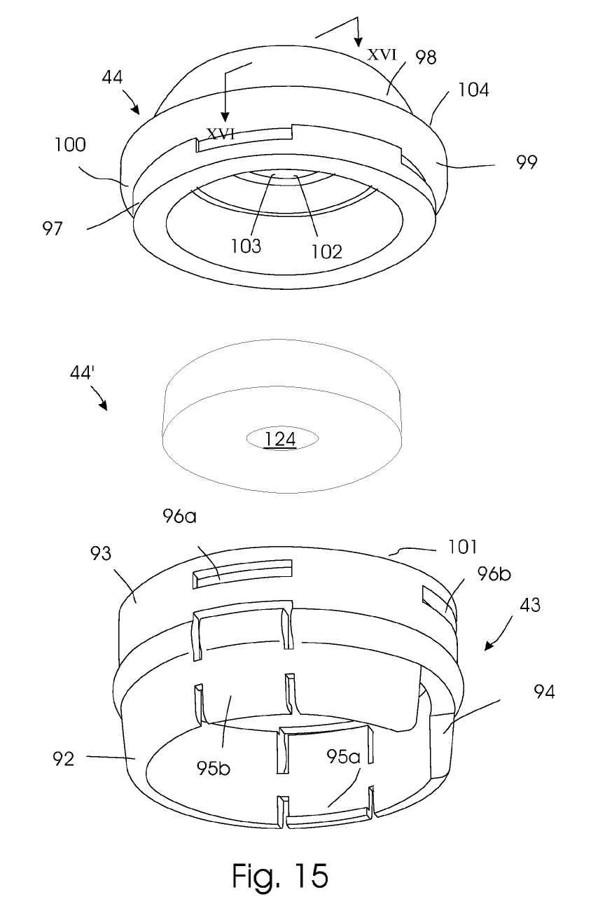

FIG. 15 shows a perspective, exploded view of a fastening ring, a guide member and a greasing ring, for mounting the valve seal according to the present invention on a trocar cannula,

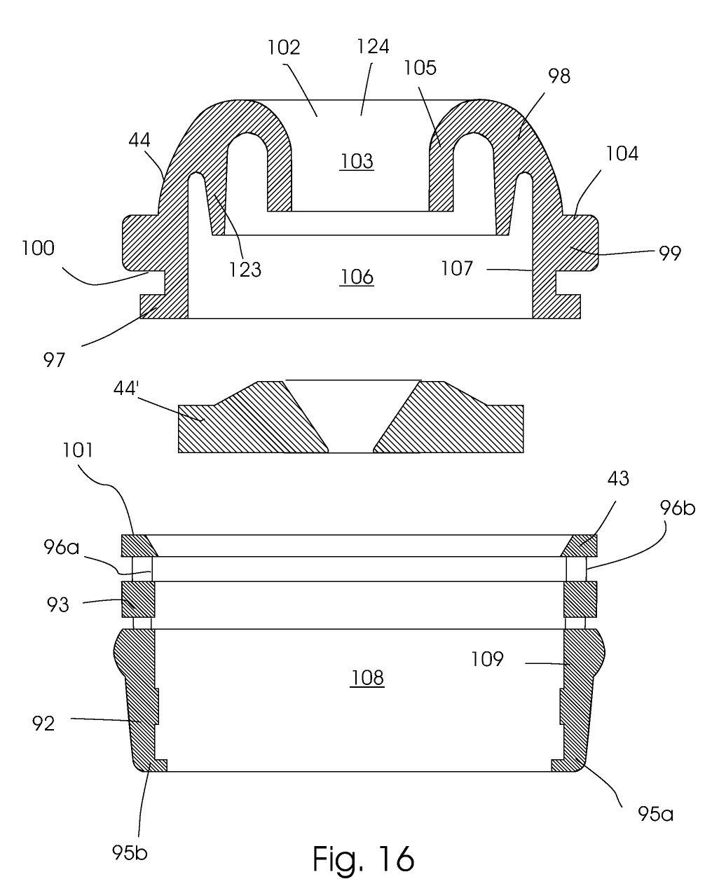

FIG. 16 shows in an enlarged scale a sectional view taken along line XVI-XVI in FIG. 15

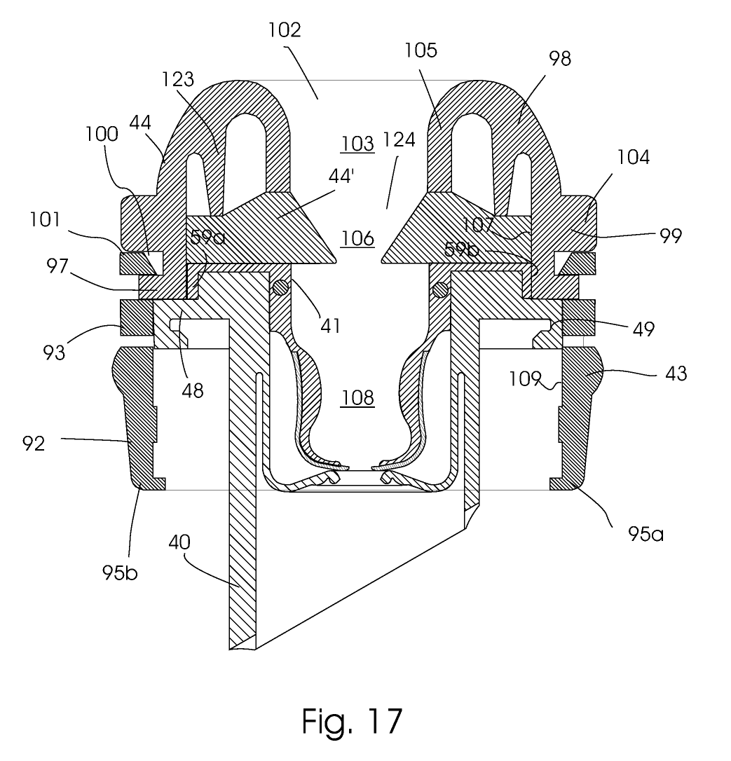

FIG. 17 shows in an enlarged scale the sectional view of the second embodiment of a valve seal fitted with the second embodiment of a friction reducing cup as illustrated in FIG. 14 further fitted with the assembled fastening ring, the guide member and the greasing ring,

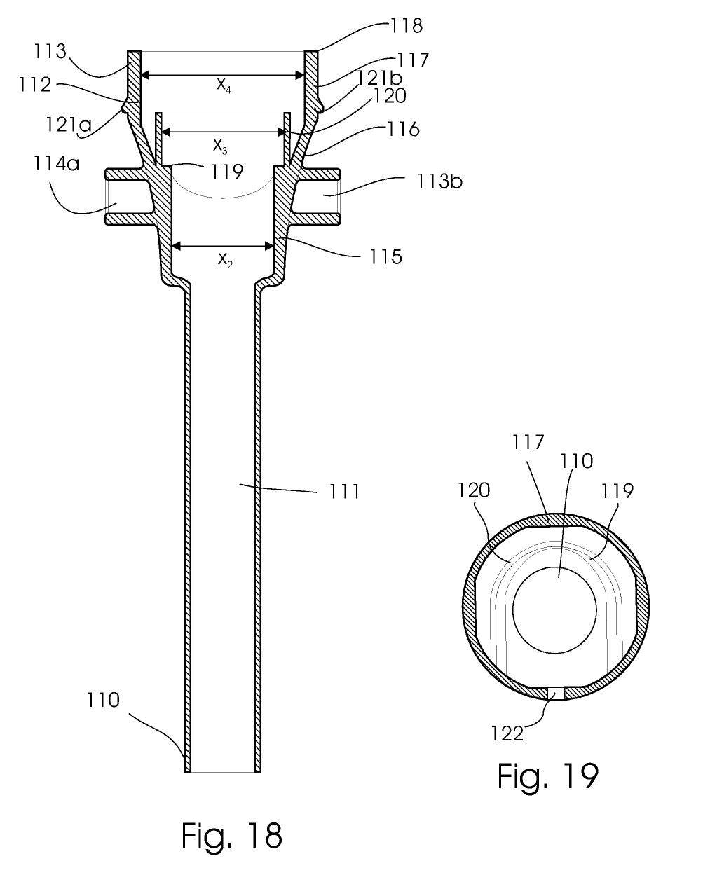

FIG. 18 shows an axial sectional view taken along line XVIII in FIG. 8 of the trocar cannula according to the present invention, and

FIG. 19 shows a cross-sectional view taken along line XIX-XIX through the enlarged section of the trocar cannula shown in FIG. 8.

FIG. 1 shows in perspective a blank 1 which can be transformed into a valve seal for a trocar. FIG. 2 shows the same seen in section. FIGS. 1 and 2 will be described in conjunction below in order to clearly describe the internal and external structure of the valve seal according to the present invention. In the following description of FIGS. 1 and 2 it is assumed, as an example, that a valve seal is made from a blank of an elastomeric material and that the central opening of the first diaphragm valve has a greater diameter that the central opening of the second diaphragm valve. However, if the elasticity of the selected material allows for it the diameters could also be substantially of similar size.

The blank 1 has a tubular neck part 2, defined by a first circumferential wall 3, an instrument receiving end 4 with a first diaphragm valve 5, and a sloped instrument leavning end 6. The first diaphragm valve 5 extends radially into a circumferential flange 7, and the circumferential flange 7 further extends axially into a tubular head part 8 defined by a second circumferential wall 9 provided with three circumferential ribs 10,11,12. The circumferential flange 7 extends into the tubular head part 8 via a pivot joint 13, in the present case a section of the second circumferential wall 9 of reduced wall thickness.

The first circumferential wall 3 of the tubular neck part 2 is split into an exterior tubular neck part 14 and an interior tubular neck part 15. The circumferential gap 16 between the exterior 14 and interior 15 tubular neck parts allows an instrument (not shown) to be pushed around inside the valve seal.

The interior tubular neck part 15 has a free end 17 provided with a second diaphragm valve 18, said second diaphragm valve 18 has a valve opening 19 which is provided with an annular bead 20. The valve opening 19 of the second diaphragm valve 18 tapers towards the instrument receiving end 4 and the opening 21 of the first diaphragm valve 5.

The blank 1 constitutes a funnel-shaped preform traversed by two diaphragm valves 5;18 situated at a distance from each other. The diameter of the central opening 21 of the first diaphragm valve 5 is in FIG. 2 shown to have a greater diameter than the central opening 19 of the second diaphragm valve 18, to thereby facilitate insertion of the instrument. The second diaphragm valve 18 is substantially cone-shaped and tapers towards the first diaphragm valve 5. When an instrument passes through the central opening 19 to reach a target, the central opening 19 is pressed axially in the same direction as the instrument resulting in that the cone is flattened so that the central opening 19 tightens around the instrument. An instrument having the same diameter as the central opening 21 of the first diaphragm valve 5, can easily pass through a central opening 19 of the flexible second diaphragm valve 18 having a smaller diameter, because the wall of the interior tubular neck part can be pushed into the radial clearance provided by the gap 16 arising from splitting up the first circumferential wall 3 of the tubular neck part 2. In addition, the central opening 19 of the second diaphragm valve 17 is expanded due to the radial force applied by the instrument, so the cone-shape of the second diaphragm valve together with the gap 16 allows an instrument to be moved in and out and from side to side in the valve seal without loss of sealing around the instrument. Accordingly, the valve seal is not susceptible to loss of sealing capability around an inserted instrument even if this instrument is moved and displaced to a considerably extent.

FIG. 3 shows an exploded view of a first embodiment of a preferred valve seal 22 according to the present invention. FIG. 4 shows the same seen in section. FIGS. 3 and 4 will be described below in conjunction in order to clearly describe the internal and external structure of the valve seal 22 according to the present invention.

The valve seal 22 may be made by folding a blank 1 as described above, or may be made in its preset form directly, and for like parts same numerals are used.

The valve seal 22 is equipped with a third valve 23 for keeping the instrument leaving end 6 of the valve seal 22 closed prior to inserting an instrument and after removal of the instrument. Further, the valve seal 22 is equipped with means 24 for reducing friction between the valve seal and the instrument when the instrument is moved in and out of the valve seal 22. The means for reducing friction is a cup 24 having a cylindrical body 25 and a partly closed bottom 26, from which a number of slits 27 radiate. In the present case six slits 27 radiate. The cup 24 is inserted between the first diaphragm valve 5 and the second diaphragm valve 18 of the valve seal 22 to provide a smooth sliding of an instrument in the valve seal 22. The smooth sliding enables the surgeon to make a more accurate positioning of an instrument inside the body, and makes the surgical procedure more comfortable and painless for the patient. An advantageously side effect is that the cup 24 acts like a back stop for preventing unintended withdrawal of the second diaphragm valve 18 towards the first diaphragm valve 5 when the instrument is withdrawn, as seen in FIG. 5.

The main structure of the third valve 23 is known per se from e.g. International patent application no. WO 01/91834. The third valve has a ring 28, which is connected to a hinged flap 29 by means of leg 30. The leg 30 forms an angle to the flap of substantially the same degree as the angle of the sloping instrument leaving end 6 in relation to the longitudinal axis of the valve seal or blank. The angle may be e.g. 45°, but other sizes of angles are within the scope of the present invention.

The third valve 23 is mounted around the tubular neck part 2 to firmly abut the side 33 of the inverted tubular head part 8 facing the instrument leaving end 6 in frictional or slightly squeezing engagement in the space 32.

The flap 29 has a convexity 31 pointing towards the central opening of the second diaphragm valve 18. Preferably, the valve seal 22 is dimensioned so that when the flap 29 abut and closes the sloped instrument leaving end 6 the convexity 31 of the flap 29 abut a annular rib 34 on said end, to enhance closing efficiency. When no instrument is inserted the pressure from gasses or liquid inside the body causes the flap to cover and close the instrument leaving end 6. The convexity 31 and the length of the interior tubular neck part 15 may be chosen so that when the central opening 19 of the second diaphragm valve 18 covers and closes this opening 19, the convexity also closes the central opening 19 of the second diaphragm valve 18 to thereby double the surgical safety and unintentional leakage from the body through the trocar cannula when no instrument is inserted.

The valve seal 22 has a tubular neck part 2 similar to the one described with reference to FIGS. 1 and 2, in which the second circumferential wall 9 of the tubular head part 8 is folded or pivoted at the pivot joint 13 to thereby expose the first diaphragm valve 5 and the circumferential flange 7. The ribs 10,11,12 is forced to face towards the circumferential wall 3 of the tubular neck part 2 as is more clear from FIG. 4, to defined a space 32 for accommodation of the wall of the cannula when the valve seal 22 is to be mounted on said cannula. Preferably, the valve seal is made of an elastomeric material so that said mounting is made in a manner substantially similar to application of a rubber band, thereby enabling the valve seal 22 to be attached without additional means.

As seen in FIG. 5 the friction reducing cup 24 is inserted in the valve seal 22 confined between the first diaphragm valve 5 and the second diaphragm valve 18 in contact with said valves 5;18.

FIG. 6a shows the valve seal 22 seen from above and FIG. 6b from below.

FIG. 7 shows a second embodiment for a valve seal 34, which only differs from the first embodiment for a valve seal 22 in that the flap 35 of the third valve 23′ is mounted as an integrated part of the valve seal 33. The instrument leaving end 6 of the tubular neck part 2 has a first engagement means 36, e.g. a rib, for coupling with a complementary shaped second engagement means 37, e.g. a groove, on the flap, so as to obtain a tight fit between the flap and the instrument receiving end. The backpressure from the gasses or liquids inside the body forced the engagement means 36,37 against each other and into engagement. The engagement means can e.g. be a snap-fitting or a force-fitting or simply since the first and the second coupling means 36,37 are made of resilient material they easily conform to each other. Alternatively, just one of the instrument leaving end 6 or the flap 35 has a protrusion serving as a packing ring.

FIG. 8 shows a perspective exploded view of a trocar 38 without a stylet. The main parts of the trocar 38 without the stylet include a trocar cannula 39, fitted with a third embodiment for a valve seal 40, a second embodiment for a friction reducing cup 41 consisting of two separate parts 41a,41b, a third valve 42, an annular fastening ring 43, an annular guide member 44 for a surgical instrument (not shown), and a greasing ring 44′ for greasing the instrument during operation.

These main parts 39,40,41,42,43,44,44′ will be described in more detail in the following with reference to the enlarged scale perspective and sectional views of the following FIGS. 9-19.

FIG. 9 shows the third embodiment for a valve seal 40 and a friction reducing means 41 oblique from an instrument leaving end 45. The major part of the internal structure of the third embodiment of the valve seal 40 corresponds substantially to the first and second embodiments, and for advantages and main function reference is made to the description of these embodiments.

The valve seal 40 has an instrument leaving end 45, an opposing instrument receiving end 46, a tubular neck part 47, which via a radial circumferential flange 48 extends into a second circumferential wall 49 concentric with the first circumferential wall 50 of the tubular neck part 47, thereby defining the opening 53 of the instrument receiving end 46. The instrument receiving end 46 of the tubular neck part 47 has an axial annular extension 52 surrounding the opening 53. Further, the second circumferential wall 49 ends, in the embodiment shown, in a single circumferential rib 54 facing towards the first circumferential wall 50. Opposing, axially extending grooves 55a,55b are recessed in the exterior face of the second circumferential wall 49. The instrument leaving end 45 is sloped as described for the first embodiment for a valve seal 22.

The annular extension 52 serves for mounting of the friction reducing cup 41, the circumferential rib 50 serves for mounting of the third valve 42, the grooves 55a,55b serves for mounting of the fastening ring 43 on the trocar cannula 39, and the fastening ring 43 serves for mounting of the guide member 43.

The friction reducing cup 41 consists in the case shown of two separate parts, a first part 41a and a second part 41b partly joined in overlapping relationship along the longitudinal axis A of the valve seal 40 and the friction reducing cup 41. More than two part are foreseen within the scope of the present invention. The first part 41a consists of a first bottom part 56a, a first central part 57a, and a first flange part 58a. The second part 41b consists of a second bottom part 56b, a second central part 57b, and a second flange part 58b. The first flange part 58a and the second flange part 58b together define the annular flange 58. The flange 58a,58b has a circumferential collar 59a,59b, which defines an annular, groove 60 fitting on top of the complementary shaped axial annular extension 52 of the valve seal 40. Thus, the flange 58 of the friction reducing cup 41 is seated on top of the axial annular extension 52 of the valve seal 40, so that this axial annular extension 52 protrudes inside the groove 60 to prevent the friction reducing cup 41 from being axially and radially displaced during operation of the trocar and surgical instrument.

The structural design by means of which the first bottom part 56a and the first central part 57a are axially and/or radially insertable into the second bottom part 56b and the second central part 57b is seen more clearly in FIG. 10.

The largest external diameter D₁ of the first bottom part 56a and the largest external diameter D₂ of the first central part 57a, respectively, are smaller than or equal to the largest internal diameter d₁ of the second bottom part 57b and the largest internal diameter d₂, respectively, enabling the first bottom part 56a and the first central part 57a to fit smoothly inside the second bottom part 57b and the second central part 57b. Due to the flexibility of both the circumferential wall of the first bottom part 56a and the first central part 57a and the circumferential wall of the second bottom part 57b and the second central part 57b, the first bottom part 56a and the first central part 57a conform easily inside the second bottom part 57b and the second central part 57b upon axially displacement, and optionally radially displacement, of the first bottom part 56a and the first central part 57a when the first part 41a is located inside the internal cavity 61 of the second bottom part 56b and the second central part 57b. The outside face 62 of the bottom 63 of the first bottom part 56a then rests on the inside face 64 of the bottom 65 of the cavity 61 of the second bottom part 56b arranging the bottom opening 66 of the first bottom part 56a aligned with the bottom 67 opening of the second bottom part 57b.

The exterior face of the circumferential wall section 68 of the first bottom part 56a and the first central part 57a is annularly recessed to be accommodated into a corresponding annular wall section 69 of the second bottom part 56b and central part 57b so that a smooth exterior and interior face of the combined first part 41a and second part 41b can be achieved to prevent an instrument tool tip from getting stuck inside the friction reducing means.

The axial face of the first flange part 58a has a projection 70 fitting into a corresponding hole 71 on the axial face of the second flange part 58b. The axial face of the first flange part 58a further has a hole 72 for receiving a corresponding projection 73 on the axial face of the second flange part 58b. One or more projection and holes may be provided on any section of the axial faces of the flange parts 58a,58b, including the annular sleeves 74a,74b connecting the central parts 57a,57b with the flange parts 58a,58b, and the collar parts 59a,59b.

When the first part 41a is assembled with the second part 41b, these parts are turned towards each other in the directions indicated with the arrows B₁ and B₂, so that the first bottom part 56a and the first central part 57a are axially inserted into the cavity 61. When the bottom 62 of the first bottom part 56a abuts the inside face 64 of the bottom 65 of the cavity 61 of the second bottom part 56b the first part 41a are axially in place, and the sleeves 74a,74b and the flange parts 58a,58b are joined by means of the projections 70,73 which fit into the corresponding holes 71,72.

In a preferred embodiment the circumferential wall section 68 of the first part 41a, which is recessed, and the corresponding annular wall section 69 of the second part 41b extends axially until the sleeves 74a,74b, which sleeves preferably have an axial length of about the same as the collars 59a,59b, to thereby provide an overlap of the entire centrals parts 57a,57b and bottom parts 56a,56b.

FIG. 11 shows in perspective the second embodiment of a third valve 42 for keeping the instrument leaving end 45 of the valve seal 40 closed prior to inserting an instrument and after removal of the instrument. The third valve has a ring 28, which is connected to a hinged flap 75 by means of a leg 76. The leg 76 forms an angle to the flap 75 of substantially the same degree as the angle of the sloping instrument leaving end 45 in relation to the longitudinal axis A of the valve seal 40. The angle may be e.g. 45°, but other sizes of angles are within the scope of the present invention.

The flap 75 of the third valve 42 is specifically designed and dimensioned so that for example an L- or J-shaped tool tip is unable to grasp the hinged flap 75 upon withdrawal of the tool. A convexity 77 is depressed in the bottom face 78 of the flap 75 opposite the ring 28 thereby providing an elevated protrusion 79 against the ring 28. A tool tip hitting the elevated protrusion 79 during inserting an instrument deflects the flap 75 and increases the angle between the flap 75 and the leg 76. As long as the instrument is inserted the increased angle is maintained and the instrument leaving end 45 of the valve seal 40 is kept open. The elevated dome-shaped protrusion 79 extends opposite the top of the dome into a rim section 80 at least a part which has an annular bead 81 along the perimeter. The protrusion 79 and the bead 81 extends in the case shown on both sides of a centre plane through the flap as seen more clearly in the sectional view of FIG. 12 taken along line XII in FIG. 11. The protrusion 79 and the bead 81 limits the radial deflection of the flap 75 and a hook-shaped tool tip cannot get beneath the flap and catch it upon withdrawal of the instrument.

The ring 28 of the third valve 42 is mounted around the tubular neck part 47 below the circumferential flange 48, e.g. by means of frictional force. The leg 76 extends and abuts the exterior face of the tubular neck part 47 allowing the flap 75 to close the instrument leaving end 45 when no instrument or stylet is inserted. The bottom edge 82 at the instrument leaving end 45 of the tubular neck part 47 forms a lip which then rests on top of the rim section 80, to thereby allow the protrusion 79 to extend into the sloped instrument leaving end 45 to close this end.

FIG. 12 shows a sectional view through the flap 75, illustrating the depths of the convexity 77. For instruments provided with a tip of app. 5 mm typically the dome protrudes app. 4 mm from the centre plan, illustrated by the line C in FIG. 12 towards the second diaphragm valve 87, and the annular bead protrudes oppositely from the centre plan C a substantially equal distance. The width of the flap is indicated to be the size x₁.

FIG. 13 illustrates the internal design of the third embodiment of a valve seal 40 and the friction reducing cup 41.

The first circumferential wall 50 of the tubular neck part 47 is split into an exterior tubular neck part 83 and an interior tubular neck part 84. The circumferential gap 85 between the exterior 83 and interior 84 tubular neck parts allows an instrument (not shown) to be pushed around inside the valve seal 40.

The interior tubular neck part 47 has a free end 86 provided with a second diaphragm valve 87, said second diaphragm valve 87 has a valve opening 88 which is provided with an annular bead 89 and a annular section 90 of increased thickness h concentric with the bead 89. The second diaphragm valve 87 tapers towards the opening 53 of the instrument receiving end 46 into which the friction reducing cup 41 is to be inserted to define the first diaphragm valve 51. Instrument diameters as large as even 12 mm pass easily through the first diaphragm valve 51. The annular section 90 of increased thickness and the bead 86 constitutes a radial flexible bellow facilitating further advancement of the instrument through the more narrow second diaphragm valve 87, to thereby allow the instrument to easily pass on further through the valve opening 88 of the second diaphragm valve 87 and seal in a reliable manner.

As also seen in FIG. 13 the second embodiment of a friction reducing cup 41 is assembled of the first part 41a and the second part 41b. The first bottom part 56a and the first central part 57a of the second part 41b are surrounded by the second bottom part 56b and the second central part 57b of the second part 41b to define a splitted-up friction reducing cup where contacting, overlapping circumferential walls are allowed to open and close freely in response to external forces and memory shape of material and design.

As is clear from the sectional view of the friction reducing cup 41, the overlap does not in the embodiment shown extend along the whole axial length below the sleeves 74a,74b, however a full overlap extending from the distal lower edge of the sleeves to the bottoms 63,65 is contemplated within the scope of the present invention.

Such a long axial and radial overlap may provide an even higher degree of flexibility due to the fact that a longer length can open to a higher degree in relation to each other. The internal diameter d₃ at the constriction 91 of the first central part 57a of the first part 41a is smaller than the largest internal diameter d₄ of the bottom part 56a of the first part 41a. The constriction 91 serves for guiding the instrument and effectively seal around said instrument. Because the largest internal diameter d₄ inside the bottom part 56a is larger than the diameter d₃ at the constriction 91 the bottom openings 64,66 are allowed to expand to increase the opening diameter by pushing the circumferential walls of the first and second central parts and the first and second bottom parts radially away from each other in response to penetration of the instrument.

In FIG. 14 the second embodiment of the friction reducing cup 41 is inserted into the valve seal 40 with the openings 64,66 of the friction reducing cup above the opening 88 of the second diaphragm valve 87 and abutting said second diaphragm valve. The opening 53 of the valve seal 40 is arranged substantially concentric with the opening of the friction reducing cup 41 to define the first diaphragm valve 51.

This specific design of the friction reducing cup 41 provides a circumferential gap 105 between the second circumferential wall 49 and the exterior face of the friction reducing cup 41. The instrument is inserted and advanced through the first diaphragm valve 51, the bottom opening 66 of the friction reducing cup 41, and the second diaphragm valve 87 and exits the third embodiment of the valve seal 40 through the instrument leaving end 45 to reach its specific targets inside the body. When the tip of the instrument is manoeuvred around in substantial circular movements the circumferential wall section 68 of at least the first bottom part 56a and the first central part 57a and the annular wall section 69 of at least the second bottom part 56b and central part 57b is able to radially deflect into the gap in the same direction as the tip of the instrument is directed. Due to a combination of structural features such as rigidity provided to the second diaphragm valve 87 by means of the bead 89 and the annular section of increased thickness 90, and the lack of venting of the gap due to the sealing capability of the second diaphragm valve 87, the deflected annular walls 68,69 pulls along the second circumferential wall 49 in the same direction as the tip of the tool is directed. The second circumferential valve 49 of the valve seal 40 and the circumferential walls 68,69 act as an integrated unit having a very high degree of radial flexibility without compromising the sealing properties. Hence, the second embodiment of a valve seal for at trocar provides the surgeon with a hitherto unknown freedom to operate a surgical instrument in a minimal invasive or laparoscopic surgical procedure.

In order to prevent the second embodiment of a friction reducing cup 41 to slip into the valve seal 40 upon manoeuvring an instrument, a fastening ring 43, a guide member 44 and if preferred a friction reducing ring 44′ are used for surrounding and holding the friction reducing cup 43 in situ with the flange parts 58a,58b firmly on top of the an axial annular extension 52.

Exemplary embodiments of a fastening ring 43, a guide member 44 and a greasing ring 44′ suitable for use with a trocar cannula and the valve seals according to the present invention is seen in perspective in FIG. 15. The material of which the fastening ring 43 and a guide member 44 are manufactured are preferably a rigid plastic material in contrast to the valve seal and the friction reducing means which are both made as flexible plastic components. The material of the greasing ring is selected to be of a kind which is easy to grease with glycerine and has an inherent compressibility, e.g. a rubbery material such as PVA (polyvinyl acetate).

The fastening ring 43 has a first coupling end part 92 for the cannula 39 and an opposing second coupling end part 93 for the guide member 44. The first coupling end part 92 has a U shaped cut-out section 94 to provide a space for allowing passage of a protruding insufflation gas inlet means on the trocar cannula 39. Further the first coupling end part 93 has means 95a,95b for coupling to the trocar cannula. In the embodiment shown the means 95a,95b are L-shaped, flexible hooks, which are snap-fitted below corresponding protrusions on the trocar cannula. The shape and number of means for coupling the fastening ring to the trocar cannula may vary within the scope of the present invention to achieve the secure coupling together of the fastening ring 43 with the trocar cannula 39. The second end part 93 serves for coupling with the guide member 44 and has in the case shown four, annularly, evenly distributed rectangular openings 96a,96b,96c,96d. The openings are optional but can be used to get access to a first coupling flange 97 on the guide member 44 to uncouple the fastening ring and the guide member 44. Alternatively the openings may be used to snap-fit or couple with corresponding snap-fitting means or coupling means on the guide member. In FIG. 15 only two openings 96a,96b. As with the means for coupling the fastening ring 43 to the trocar cannula 39 also the shape and number of the opening may vary within the scope of the present invention, as long as the coupling between the fastening ring and the guide member is strong and secure enough for restraining the valve seal assembly in relation to the trocar cannula.

Further FIG. 15 shows in perspective an exemplary embodiment of a guide member 44 for coupling with the fastening ring 43. The first coupling flange 97 of the guide member 44 extends into a tapering part 98 via a second coupling flange 99 which forms an abutment face 100 for the free end face 101 of the fastening ring 43's second coupling end part 93. The dome-shaped tapering part 98 defines a fourth valve 102 with a first passageway 103 for an instrument. The fourth valve 102 may seal around certain sizes of instruments but first of all it serves as a guide for the instrument during the first stage of insertion and for directing the instrument tip towards the first diaphragm valve 51.

The greasing ring 44′ is dimensioned to fit into the guide member 44, as will be more clearly understood from FIGS. 16 and 17.

FIG. 16 illustrates the internal structural design of the fastening ring 43 and the guide member 44. The inverted wall 105 of the fourth valve 102 serves as a funnel-shaped guide during entry of the instrument tip, and during the further advance through the second clearance or passage 106 of the guide member opposite the fastening ring 43, which second clearance has a larger diameter than the first passageway 103. The internal diameter of the second clearance of the guide member is defined by the annular face 107, and the internal diameter 108 of the fastening means 43 is defined by the annular face 109.

In operational use the third embodiment for a valve seal 40, arranged as illustrated in FIG. 14, i.e. configured with the friction-reducing cup 41, is inserted to engage the annular face 109 of the internal diameter 108 of the fastening ring 43 of the fastening ring. Subsequently the guide member 44 with the greasing ring 44′ is snap-fitted to the fastening ring 43, as seen in FIG. 17, where the circumferential collars 59a,59b abut the annular face 107 of the second clearance of the guide member 44, the second circumferential wall 49 of the valve seal 40 abuts the internal annular face 109 of the fastening ring 43, and the first coupling flange 97 abuts the first circumferential flange 48 of the valve seal 40. The free end face 101 of the fastening ring 43's second coupling end part 93 abuts the second coupling flange 99 of the guide member to keep the guide member 44 in tight and secure contact to hold the valve seal 40 and the friction reducing cup 41 firmly captured between them.

The greasing ring 44′ is axially squeezed and confined between the inverted wall 105 of the fourth valve 102 and the flange parts 58a,58b of the friction reducing cup 41. The axial down-squeezing is increased along the perimeter of the greasing ring 44′ by means of an annular internal wall 123. The through-going opening 124 of the compressible greasing ring 44′ is made conical by means of the depression and squeezing obtained by the annular internal wall, and this encourage the greasing ring to reduce the diameter of the through-going opening at least towards the first diaphragm valve. Alternatively, the greasing ring may be premade with a conically tapered through-going opening. This design ensures that the instrument always contact the greasing ring.

FIG. 18 shows an axial sectional view of the trocar cannula 39 shown in FIG. 8 taken along line XVIII-XVIII. The trocar 39 has a distal end 110, which is inserted through e.g. an incision in the abdominal wall. The distal end 110 extends via a cannula part 111 into an opposing proximal end 112 extending into an enlarged section 113 for accommodating at least a part of the valve seal 22;34;40. The proximal end 112 has opposing hand grips 114a,114b for holding and operating the trocar during and after insertion into the body. The enlarged section 113 consist of a lower part 115 emerging from the cannula part 111, and an intermediate part 116 emerging from the lower part 115 into an upper part 117 ending in a free end 118. The lower part has an annular external extent, which is larger than the external diameter of the cannula part 111 to ensure that only the cannula part 111 is inserted into the body. Hence, the enlarged section 113 serves as a penetrational stop. The bore of lower part 115 is substantially tubular with an internal diameter x₂ that corresponds to or is smaller than the width x₁ of the flap 75 allowing this flap to pass axially and radially at least a distance into this tubular lower part 115 when displaced by an instrument. The tubular lower part 115 opens gradually into a funnel-shaped intermediate part 116 via an oblique semicircular shoulder 119 for controlling displacement of the flap 75. An U-shaped wall 120 projects from the shoulder 119 inside the intermediate part 116 towards the upper part 117. The width x₃ between the legs of the U is larger than the internal diameter x₂ of the tubular lower part 115, and smaller than the internal diameter x₄ of the upper part 117, to accommodate the valve seal, the friction reducing cup, and the third valve in a manner which allows the flap of the third valve to be displaced. The U-shaped wall 120 restricts the radial movement of an inserted instrument. When a tip with a hook, such as an L- or J-shaped hook, is withdrawn through the instrument leaving end 45 of the valve seal 40, there exists a risk that the hook catches the flap 75 of the third valve 41. This risk is dramatically reduced or even eliminated by means of the U-shaped wall 120 of the enlarged section 113, due to the fact that the U-shaped wall 120 restricts the radial movement of the tool, preventing the hook from getting around the flap 75. At the transition between the intermediate part 116 and the upper part 117 the exterior face of the enlarged section 113 has opposing coupling protrusions 121a,121b for coupling with the first coupling end part 93 of the fastening ring 43. The L-shaped, flexible hook means 95a,95b snap-fits into engagement with the coupling protrusions 121a,121b on the enlarged section 113 of the trocar cannula 39.

The cross-sectional view of FIG. 19 taken along line XIX-XIX of FIG. 8 of the intermediate part 116 shows the U-shaped wall 120 and the annular shoulder 119 from the proximal end 112 of then trocar cannula 39 down into the enlarged section 113. The sectional view is taken through the gas inlet opening 122, which may or may not be provided with means for opening and closing the supply of an insufflation gas.

The valve seals according to the present invention are not limited to use with third valves designed with flaps, and in a very simple embodiment the third valve is also a diaphragm valve, traversing a sloped or non-sloped instrument leaving end.

The design of the valve seals according to the present invention is suitable for trocars with different diameters, thus for use with instruments with diameters of different sizes, e.g. diameters ranging from 5-12 mm, but the same design can also be used for valve seals for trocars for instruments or apparatus' having greater or smaller diameters.

The blank may within the scope of the present invention be made with a third valve corresponding to the integrated flap closure means as described for the second embodiment for a valve seal according to the present invention. The flap may also be made as a separate part adapted to be hinged directly on the instrument leaving end of the first circumferential wall of the valve seal in which case the leg and the ring may be dispensed off.

The valve seals, the friction reducing means and the third valves together forms valve seal assemblies with improved flexibility and sealing when an inserted surgical instrument is operated.

1-32. (canceled)

33. A valve seal for a trocar cannula having a distal end and an opposing proximal end with an enlarged section for accommodating at least a part of a valve seal, wherein the valve seal has:
a tubular neck part defined by a first circumferential wall, an instrument leaving end and an opposing instrument receiving end,
the instrument receiving end is provided with a first diaphragm valve and a circumferential flange connecting the tubular neck part with a substantially concentric second circumferential wall surrounding at least a part of the first circumferential wall to define a gap between said substantially concentric circumferential walls,
at a distance from the first diaphragm valve the first circumferential wall of the tubular neck part is axially split up into an exterior tubular neck part and a substantially concentric interior tubular neck part provided with a second diaphragm valve, said interior tubular neck part terminates a distance from the instrument leaving end.

34. The valve seal according to claim 33, wherein at least one of the first and second diaphragm valves have an annular section of increased thickness or a bead along or surrounding the perimeter of its respective central openings.

35. The valve seal according to claim 33, wherein at a face of the second circumferential wall facing towards the tubular neck part the second circumferential wall is provided with at least one coupling rib or thread.

36. The valve seal according to claim 33, wherein the instrument leaving end of the exterior tubular neck part is sloped.

37. The valve seal according to claim 33, which further comprises a third valve attached to the valve seal or is an integrated part of the valve seal, wherein the third valve has a hinged flap which is displaceable to open the instrument leaving end of the valve seal by means of an instrument introduced in the valve seal and to substantially close the instrument leaving end when the instrument is retracted from the valve seal.

38. The valve seal according to claim 37, wherein the flap has a convexity facing the instrument leaving end of the valve seal.

39. The valve seal according to claim 33, wherein the valve seal further comprises means for reducing friction between the valve seal and the instrument when the instrument is moved in and out of the valve seal, at least a part of the friction reducing means is inserted between the first diaphragm valve and the second diaphragm valve.

40. The valve seal according to claim 39, wherein the means for reducing friction is a cup wherein at least a bottom part is split-up.

41. The valve seal according to claim 40, wherein the bottom part of the cup extends axially via a central part into a circumferential flange.

42. The valve seal according to claim 41, wherein the smallest internal diameter of at least a first axial section of the central part closest to the bottom part is smaller than the largest internal diameter of the bottom part itself.

43. The valve seal according to claim 42, wherein the smallest external diameter of at least the first axial section of the central part closest to the bottom part is smaller than the largest external diameter of the bottom part itself.

44. The valve seal according to claim 39, wherein the means for reducing friction is a cup comprising:
a first part having at least a first bottom part and a first central part, and
a second part having at least a second bottom part and a second central part,
wherein the first bottom part is insertable into the second bottom part or vice versa.

45. The valve seal according to claim 44, wherein the first central part is insertable into the second central part or vice versa.

46. The valve seal according to claim 44, wherein the friction reducing cup further comprises that the first central part extends into a first flange part and the second central part extends into a first flange part, wherein the first and second flange part have coupling means for annular coupling.

47. The valve seal according to claim 44, wherein the valve seal and friction reducing cup are moulded of an elastomeric material as an integral unit.

48. The valve seal according to claim 44, wherein at least one of the valve seal or the friction reducing cup or both are moulded of a material having self-lubricating properties.

49. A trocar cannula having a distal end and an opposing proximal end with an enlarged section for accommodating the valve seal according to claim 33, wherein the trocar cannula further comprises a fastening ring having a first end part for coupling with the enlarged section of the proximal end of the trocar cannula and an opposing second end part.

50. The trocar cannula according to claim 49, which further comprises an instrument guiding member detachable coupled to or being an integrated part of the fastening ring.

51. The trocar cannula according to claim 50, wherein the instrument guiding member comprises an instrument greasing ring.

52. The trocar cannula according to claim 49, wherein the enlarged section has a gas inlet opening.

53. The trocar cannula according to claim 49, wherein at least an annular part of the enlarged section has an interior wall part to reduce the cross-section of the enlarged section.
